(19) United States
(12) Patent Application Publication
(10) Pub. No.: US 2014/0324006 A1
(43) Pub. Date: Oct. 30, 2014

(54) Wax-Containing Hot Melt Adhesives and Disposable Absorbent Articles

(71) Applicant: Rong Zhong, Guangzhou (CN)

(72) Inventor: Rong Zhong, Guangzhou (CN)

(73) Assignee: BOSTIK S.A., Courbevie (FR)

(21) Appl. No.: 14/119,750

(22) PCT Filed: May 4, 2012
(86) PCT No.: PCT/EP12/58198

§ 371 Date: Jul. 11, 2014

(30) Foreign Application Priority Data
May 23, 2011 (CN) 201110134835.2

(51) Int. Cl. A61L 15/58; C09J 157/00; C09J 145/00; A61F 13/15; A61L 15/22

(52) U.S. Cl. CPC A61L 15/585; A61F 13/15699; A61L 15/225; C09J 145/00; C09J 157/00
USPC 604/366; 524/505; 427/207.1

(57) ABSTRACT

1) Wax-containing hot melt adhesive comprising 1 to 40% of a styrenic block copolymer (a), 3 to 15% of a modified thermoplastic elastomer (b) comprising —COO— groups, and 5 to 25% of a wax (c). 2) Disposable absorbent article employing said hot melt adhesive. The article bonded by the hot melt adhesive of the present invention has excellent creep resistance and aging resistance.

The present invention relates to the field of disposable absorbent articles. In particular, it relates to wax-containing hot melt adhesives intended for disposable absorbent articles. It also relates to disposable absorbent articles comprising said hot melt adhesives.

A hot melt adhesive is an adhesive having plasticity, whose physical state changes while the chemical characteristics do not change with the varying of the temperatures within a certain temperature range. A hot melt adhesive is non-toxic and belongs to environmentally friendly chemical products. A hot melt adhesive melts and becomes a liquid by heating, which liquid is then delivered to the surface of the object to be bonded through a hot melt adhesive hose and a hot melt adhesive nozzle, the hot melt adhesive is then cooled to achieve the bond.

Many types of hot melt adhesives are available at present; a lot of the relevant formulations are also disclosed. Chinese Patent CN1186834 discloses a hot melt adhesive composition for packaging, which is a combination of typical ethylene-vinyl acetate (EVA) and paraffin, for the automatic packaging line of carton/box. Chinese Patent CN1759156A discloses a hot melt adhesive for packaging used at low temperature, which comprises EVA and/or ethylene 2-ethyl hexyl acrylate polymer, paraffin and Rosin tackifier. Chinese Patent CN101307214A discloses a hot melt adhesive having a high temperature resistance, which comprises synthetic wax with a high melting point and is used for the bonding of the automobile interior parts.

The hot melt adhesives disclosed above cannot be applied into disposable absorbent articles. This is because the hot melt adhesives used in the disposable absorbent articles of the fast consumption products have particular requirements. The hot melt adhesives of this field, according to the usage thereof, can be classified as positioning adhesives, construction adhesives, and elastic attachment adhesives.

1. Positioning hot melt adhesives need to have certain pressure sensitivity, so as to fix the sanitary napkins or diapers to the underpants or underwear;

2. Construction adhesives are used for attaching different functional layers with different compositions together, to become a complete article, such as sanitary napkins or diapers etc.;

3. Elastic attachment hot melt adhesives are used to attach an elastic material (for instance an elastic strand) to a substrate, said substrate likely to consist in particular of a surface layer, or/and a base layer, both layers being comprised in the disposable absorbent article, such that said disposable absorbent article fits to the human body and the liquid, for instance urine, will not leak. Such hot melt adhesives are not necessarily pressure sensitive, but have to possess a relatively high cohesive strength to resist the creep during retraction of the elastic material. By said creep resistance is therefore meant the ability of the hot melt adhesive which has been used to bond the elastic material under stretch to the substrate, to maintain the quality of said bond whenever the elastic material is stretched or retracted.

There exists many steps between the time the disposable absorbent articles are manufactured at the production line and the time these articles are used by the consumers. These steps comprise transport, delivering to the supermarket, etc. The hot melt adhesives for such industry therefore have to be excellent in aging resistance, in particular with respect to creep resistance.

Chinese Patent CN1261617A discloses a hot melt adhesive for multipurpose disposable absorbent articles, which however does not comprise wax and the main polymer is limited to SBS only. This hot melt adhesive can be used as construction adhesives and elastic attachment adhesives simultaneously, but the creep resistance thereof when being used to bond elastic materials is not high. CN1918257A discloses an elastic attachment hot melt adhesive used in non-woven fabric, that hot melt adhesive has to contain ionomer resin, such ionomer resin mainly refers to the co-polymer of ethylene with acrylic or methacrylic acid which is partly neutralized by metal ions, which has a certain polarity and carries metal ions and tends to cross-link by opening the double bond of the raw material and then to polymerize again at a certain temperature. However, the polarity and metal ions thereof will lead to poor compatibility with the thermoplastic elastomer, which in turn results in poor aging resistance of the article made with this hot melt adhesive.

Aiming to overcome the deficiency of the prior art, the present invention provides a wax-containing hot melt adhesive, which improves the elastic modulus of the hot melt adhesive to achieve high creep resistance and excellent aging resistance.

To overcome the above technical problem, the present invention provides a wax-containing hot met adhesive composition which comprises, based on the total weight of said composition, the content (expressed as weight percent) of the following components:
1 to 40% of one or more styrenic block copolymer (a) with a linear or star type structure and with the general formula:

A-B-A

wherein:
A is a homopolymer of styrene or vinyl toluene or other styrene derivatives;
B is:
   butadiene and/or isoprene;
   partly hydrogenated isoprene, butadiene or isobutene;
   substantially hydrogenated isoprene, butadiene or isobutene; or mixtures thereof;
3 to 15% of a modified thermoplastic elastomer (b) comprising —COO— groups, selected in the group consisting of:
an acid or anhydride modified, linear or star type, styrenic block copolymer; and
an ethylene-vinyl acetate copolymer; and
5 to 25% of a wax (c).

The above styrenic block copolymer (a) can be used alone, or used in combination of two or more thereof.

According to a preferred embodiment, said copolymer (a) is selected among styrene-isoprene-styrene (SIS), styrene-butadiene-styrene (SBS), styrene-isoprene/butadiene-styrene (SIBS), styrene-ethylene/butylene-styrene (SEBS) or styrene-ethylene/propylene-styrene (SEPS) or star co-polymer (SI)ₙ or (SB)ₙ, wherein n is equal to or greater than 3.

Copolymer (a) may be selected among the following commercial products:
SIS Vector 4211, Vector 4114, Vector 4411 and SBS Vector 6241, Vector 4461 produced by ExxonMobil Chemical Company;
SIS D1160, D1163 and, D1124 and SEBS G1650 and G1652 produced by Kraton;
SBS1401, SBS1301 and SIS1209 produced by Yueyang Petrochemical Company;
SIS2393, SIS1307, SBS4202 and SBS3206 produced by TSRC; and
SIS3280 and SIS3345 produced by NIPPON ZEON.

The hot melt adhesive according to the present invention can use one of the SIS or SBS or SEBS, or use several copolymers in combination. It should be noted that when several copolymers are used in combination, although the styrene content of each individual copolymer before the combination could be less than 20%, the styrene content based on the total weight of styrene in the combined copolymers (a) is not less than 25%.

The content of this copolymer (a) in the hot melt adhesive composition according to the invention is preferably 10 to 30 weight %.

According to a preferred embodiment, the modified thermoplastic elastomer (b) comprised in the hot melt adhesive composition according to the invention is an organic acid or anhydride modified (more preferably grafted) SBS, SIS or SEBS. Such modified styrenic block copolymer may be prepared by conventional methods known in the art, as described for instance in the 2 following references:
article by V. L. Lasalle et al in J. Adhesion Sci. Technol., Vol. 17, N° 12, pages 1713-1726 (2003);
Chinese published patent application 1528798.

Examples of such products, which are commercially available, include the maleic anhydride modified SEBS FG1901 and FG1904 of Kraton, and the maleic anhydride grafted SBS TUFPRENE 912 produced by ASAHI KASEI.

According to another preferred embodiment, the modified thermoplastic elastomer (b) is an ethylene-vinyl acetate copolymer with a vinyl acetate content equal to or greater than 40 weight %.

As an example of an EVA (Ethylene-vinyl acetate copolymer) with the VA (vinyl acetate) content equal to or greater than 40 weight % one can cite the ELVAX 40W produced by DUPONT.

The main structure of these modified thermoplastic elastomers comprising —COO— groups is consistent with that of the main styrenic block copolymer (a), therefore such problem regarding compatibility does not exist; moreover, because the structure comprises —COO— polar groups, the bonding performance between the hot melt adhesive and the polyurethane of the elastic material is improved. As a consequence of the introduction of such organic acid modified (in particular grafted) thermoplastic elastomer into the hot melt adhesive composition according to the present invention, the bonding performance to the natural and synthetic elastic materials is maintained, while the bonding performance to elastomer polyurethane fibers, such as elastic strands, is improved due to the similar structure of the majority of the molecules and that of the elastic materials.

The content of the modified thermoplastic elastomer (b) in the hot melt adhesive composition according to the invention preferably lies in the range from 5% to 10 wt %.

According to a preferred embodiment of the hot melt adhesive composition subject of the invention, the wax (c) comprised in said composition is selected in the group consisting of: paraffin, microcrystalline wax, polyethylene wax, polypropylene wax, by-product polyethylene wax, Fischer-Tropsch synthetic wax, various modified wax and functionalized wax. The wax can be used alone, or used in combination of two or more of the waxes.

Wax is an essential component of the hot melt adhesive composition of the present invention, which contributes to significant increase of the elastic modulus of the adhesive at low temperature or ambient temperature, so as to increase the creep resistance of the hot melt adhesive. Because the wax component partly replaces the liquid plasticizer, the migration of the liquid plasticizer into the substrate during the storage of the article is decreased or prevented; this wax-containing hot melt adhesive has excellent aging resistance, in particular with respect to creep resistance.

As examples of such commercially available wax, one can cite: fully refined wax 56#, 58#, 60# and 64# (produced by No. 1 Refinery of Fushun Petrochemical Company, No. 2 Refinery of Fushun Petrochemical Company, No 7 Refinery of Dalian Petrochemical Company, SINOPEC); microcrystalline wax 80# and 68# (produced by Lanzhou Refinery and Jingmen Refinery, SINOPEC); polyethylene wax, such as L-913 and L-912 from Beijing No. 2 Assistant Factory, Licocwax PE130, PE190 and PE520 from Clariant; Epolene N21 from Eastman Chemical, etc.; polypropylene wax, such as Licocene PP1302, PP1502 and PP1602 from Clariant, A-C 8, A-C 1089 and A-C1660 from Honeywell; modified wax and functionalized wax, such as A-C325, A-C680 and A-C596 from Honeywell; SASOL WAX H105, H1, PX-100 and C80 etc.

The content of wax (c) comprised in the hot melt adhesive composition according to the invention preferably lies in the range from 5 to 15 wt %.

According to a preferred embodiment, the hot melt adhesive composition subject of the present invention further comprises 20 to 80 weight % of a tackifier resin (d).

The tackifier resin (d) is preferably selected in the group consisting of aliphatic, alicyclic and aromatic resin, and modified material and hydrogenated derivatives thereof, rosin and modified materials and derivatives of rosin, terpenes and modified materials and derivatives of terpenes. The tackifier can be one of the above resins, or can be a combination of more than one of the above resins. According to a particularly preferred embodiment, the tackifier resin (d) has a ring and ball softening point comprised between 80 and 125° C.

Such tackifier resin comprises SU400, SU210, SU420, SU100 and SU120 etc., produced by KOLON, which are partly or fully hydrogenated dicyclic pentadiene (DCPD) petroleum resin; Escorez5600\5400\5615 of EXXON CHEMICAL, which are also partly or fully hydrogenated dicyclic pentadiene (DCPD) petroleum resin; E2596\2510\2203 and E1315 etc. of EXXON CHEMICAL, which are non-hydrogenated petroleum resin; P-100, S-100 and P-125 etc. from Idemitsu Petrochemical, which are hydrogenated petroleum resin; FM-100, FM-115 and FP-125 from Formosan Union Chemical Corp., which are hydrogenated C9 petroleum resin; KF398 and KF392 from Guangzhou Komo co. ltd., which are rosin glycerol ester; KA-100L, KF452, KF454 and KF462 from Guangzhou Komo co. ltd., which are rosin ester resin; ZT5100 from Arizona, which is styrene modified terpene; P-195 and S-195 from NIPPON ZEON, which are non-hydrogenated C5/C9 petroleum resin; GA-100 and GA-90 from Wuzhou Arakawa Chemical Industries Ltd., which are modified rosin, and common premium rosin made in China, which are natural and/or synthetic thermoplastic resin with a molecular weight of 3000 or less. The selection and combination of these tackifier resins depends on the styrenic block copolymer (a) and modified thermoplastic elastomer (b) which are used.

The contents of the tackifier resin in the hot melt adhesive composition according to the present invention preferably lies in the range of from 25 to 65 wt %.

According to a preferred embodiment, the hot melt adhesive composition subject of the present invention further comprises 5 to 40 weight % of a liquid plasticizer (e).

The plasticizer (e) is preferably selected in the group consisting of paraffinic oil, naphthenic oil, aromatic oil, low molecular weight polyisobutylene, tributyl phosphate, dioctyl phthalate, liquid hydrocarbon resins. Plasticizer (e) consists more preferably of naphthenic oil.

As examples of commercially available plasticizer, one can cite: naphthenic oil KN4010, KDN100 and paraffin base oil 150BS from Karamay refinery; white mineral oil Primol N382 produced by EXXONMOBIL CHEMICAL; naphthenic oil Nyflex 222B and 223 etc. from NYNAS; dioctyl phthalate from Tianjin Chemical Reagent Second Factory; tributyl phosphate from Reagent No. 1 Factory Of Shanghai Chemical Reagent Co., Ltd.; polyisobutene with a molecular weight of 2000 to 5000 from Jinxi Refinery; liquid resin, such as Escorez 2520 from EXXON CHEMICAL, Wingtack 10 from Goodyear, and R1010 from Eastman.

The content of liquid plasticizer (e) in the hot melt adhesive composition according to the present invention lies preferably in the range of from 12 to 30 wt %.

According to a preferred embodiment, the hot melt adhesive composition subject of the present invention further comprises 0.1 to 2 weight % of an antioxidant (f).

The antioxidant (f) is selected in the group consisting of: high molecular hindered phenol, sulfur or phosphorus containing phenol, UV absorbers. Several antioxidants can be used in combination. Generally, antioxidants are used in the hot melt adhesive composition in order to protect the adhesives from being degraded due to their reaction with oxygen resulting from heat, light or induction of the residual catalyst in the tackifier resin raw materials; moreover, the antioxidants can also protect the raw material from being damaged due to the shear stress generated by high-speed mixing or kneading during the processing and using of the adhesive. As examples of commercially available antioxidants (f), the following products may be cited: Irganox 1010 and Irganox 1076 of 4,4′-methylene bis(2,6-tert-butyl phenol) type from CIBA; Weston 619 of di-n-octadecyl-3,5-di-tert-butyl-4-hydroxy-benzyl phosphate type from FAI; Sumilizer TPD of 2-(n-octyl-thio)-ethyl-3,5-di-tert-butyl-4-hydroxy benzoic acid esters type from Sumitomo; UV absorber Tinuvin P from FAI; 2,6-t-butyl-p-cresol produced by Shenyang No. 2 Reagent Factory.

The content of the antioxidants in the hot melt adhesive composition according to the present invention is preferably in the range of from 0.5% to 1 wt %.

According to a further preferred embodiment of the invention, the hot melt adhesive composition consists of:
1 to 40 weight % of styrenic block copolymer (a);
3 to 15 weight % of modified thermoplastic elastomer (b);
5 to 25 weight % of wax (c);
20 to 80 weight % of tackifier resin (d);
5 to 40 weight % of liquid plasticizer (e); and
0.1 to 2 weight % of antioxidant (f).

The hot melt adhesive composition according to the present invention is prepared as follows: the raw materials are added sequentially and melted into a homogeneous transparent adhesive solution at a temperature of about 130° C. to 180° C. Various methods of mixing or kneading known in the art, as long as they can obtain a homogenous solution of adhesive, can be used to prepare the adhesive composition of the present invention.

The present invention also provides a disposable absorbent article, which comprises a liquid permeable surface layer, a core layer for absorbing liquid and a liquid impermeable base layer, characterized in that the disposable absorbent article comprises edge sealing elastic material bonded by the wax-containing hot melt adhesive composition such as defined previously.

Said hot melt adhesive composition is used for the attaching and side bonding of the elastic material.

Such disposable absorbent articles comprise for instance baby diapers, adult incontinence products, mattresses, kids training pants, sanitary napkins and other absorbing products, such as bibs, breast pads, wound dressings and surgical caps.

The disposable adsorbent articles of the present invention comprise composite and disposable adsorbent products comprising one or more elastic materials preferably under the form of elastic strands (or fibers) or elastic films. The introduction of such an elastic material is needed in such parts as: elastic waistband, elastic leg parts or leg cuff parts.

Said elastic material may be selected among natural rubber, synthetic rubber, polyurethane, polyester-polyurethane copolymer, polyacrylate, polyether ester, styrene block copolymers, polyisoprene and other natural or synthetic materials with good elasticity. The so-called elasticity refers to the property that the material is stretched or expanded to a certain extent by external force and recovered or partly recovered when the external force is removed. Because more and more elastic materials consist of polyurethane or polyether ester, the addition of modified thermoplastic elastomer in the hot melt adhesive composition of the present invention modifies the bonding properties between the conventional elastic attachment adhesive composition and the elastic material.

The base layer material or substrate and fabric herein refer to fabric material (i.e. non-woven fabric) and the film formed without textile or weaving process. Various fibers for non-woven fabric are synthetic, natural or combination of both, wherein the fibers comprise polyethylene fibers, polypropylene fibers, polyester fibers, cotton fibers, hemp, and wool fibers, polyamide fibers, rayon fibers and acetate fibers. Such fibers for disposable absorbent article are also subjected to special chemical treatment to be hydrophilic or hydrophobic. Film material or composite of film with non-woven fabric is generally used as the base layer material of the disposable absorbent articles, this layer is impermeable but preferable permeable for steam. Such film material can be polyethylene and polypropylene films which are subjected to corona treatment, such film material can also be gas permeable films which are subjected to treatment by calcium carbonate particles.

The hot melt adhesive composition according to the present invention is used to bond the above elastic material to the above non-woven fabric and base film, to keep the disposable articles attached to the leg or waist of human body for preventing urine leakage. In the meanwhile, the hot melt adhesive composition of the present invention is also used to bond the non-woven fabric of both sides, to prevent urine from leaking. The addition of the wax in the hot melt adhesive composition according to the present invention contributes to prevent the migration of the liquid plasticizer into the micro-pores of non-woven fabric or base film, which maintains the original formulation ratio of the adhesive to obtain an excellent aging resistance.

The present invention also provides a process for manufacturing the disposable absorbent article such as defined previously which comprises the coating of the hot melt adhesive composition according to the invention:
on a substrate through a method selected among spiral spraying, fiber coating, Omega spray, Surewrap™ spray, combed slot coating, slot coating, roll coating or painting, and
at a temperature selected in the range of from 140 to 180° C.

The process for manufacturing disposable absorbent article generally requires several steps to interconnect the various components (or parts) such as: surface layer and permeable layer, permeable layer and adsorbent core layer, surface layer and base layer, in order to obtain the whole article.

Preferably, the coating is achieved through spiral spraying the hot melt adhesive composition on the elastic material as a strand.

### BRIEF DESCRIPTION OF THE DRAWINGS

FIG. 1 shows the temperature scan results of example 0, 1, 2 and 3 of the present invention;

FIG. 2 shows the creep results of example 0, 1, 2 and 3 of the present invention;

FIG. 3 shows the temperature scan result of example 0, 4 and 5 of the present invention;

FIG. 4 shows the creep results of example 0, 4 and 5 of the present invention.

### EXAMPLES

The present invention is illustrated by the following examples 1 to 5, which do not intend to limit its scope.

The hot melt adhesive compositions of the examples 1 to 5 and of the reference example 0 are further characterized by the following properties and testing methods.

1. Melt Viscosity

The melt viscosity of the hot melt adhesive composition is determined by Brookfield viscometer, type: DV-III, spindle used: 27 #.

2. Rheological Properties

The rheological properties of the hot melt adhesive composition are determined by AR2000 Rheometer of TA Instruments. The elastic modulus (G′), loss modulus (G″), glass transition temperature and cross-over temperature of the adhesive composition is obtained by temperature scan; the strain and elasticity of the adhesive composition is determined by creep testing. The instrument is controlled by software, and parallel plates with a diameter of 25 mm and distant by about 1 mm are used. Temperature scan is performed as follows: the samples are loaded at high temperature, and then the temperature decreases to 0° C., the cooling rate is 10° C./min in the range of 140˜40° C. with the strain being controlled at 5%, the cooling rate is 2° C./min in the range of 40˜0° C. with the strain being controlled at 2%, same frequency of 1 Hz is used for these two periods. The creep is determined as follows: the samples are loaded at high temperature, and then the temperature is allowed to decrease to 38° C., stabilized for 5 minutes, and then a stress of 5000 Pa is applied for 5 minutes, the stress is removed then and the samples are allowed to recover for 5 minutes.

3. Creep Resistance:

First of all, laminates are prepared through a continuous coating process in a hot melt coater for lab use produced by Hersanna Corporation Ltd by:
coating the adhesive composition to be tested at a temperature of about 160° C. on 3 parallel and stretched at 300% elongation elastic strands of Lycra® @XA 940 decitex, at a quantity of 30 mg/linear meter of elastic strand, using a spiral spray nozzle (Surewrap™ from Nordson); then
contacting said coated strands onto a first substrate consisting of a hydrophilic non-woven fabric layer; then
laminating on the bottom of the first coated substrate a second substrate of a breathable polyethylene film, the 3 elastic strands being between the first and second substrate.

After their production, these laminates are packaged by being wound up in the form of a reel.

After being held at room temperature overnight, creep resistance of these laminates is then assessed as follows.

A sample of said laminate is cut from the reel, corresponding to a length of about 350 mm, and first allowed to retract. Then the sample is stretched out completely (corresponding to an elongation of about 100%), its 2 ends being securely attached to a piece of rigid corrugated paperboard. A length of 300 mm is marked (hereafter: initial length) and the elastic strands are cut at the marks.

This sample of laminate is then placed in an air-circulating oven at 38° C. during a period of 4 hours. Under these conditions, the elastic strands under stretch can retract to a certain distance. After the 4 hours period, the distance between the ends of the sample is measured (hereafter: final length).

The creep percentage is calculated by the following formula:

Creep resistance (%)=[(initial length−final length)×100]/initial length

Five samples are tested under each condition; the results are averaged and recorded.

4. Aging Resistance:

Creep resistance is measured on samples of laminates as in §3. above, both initially (hereafter: initial creep resistance) and after allowing the samples to remain 4 weeks in an oven at 55° C. 50% RH (hereafter: creep resistance after aging).

The changing ratio of creep resistance is then calculated by the following formula:

$$changing ratio of creep properties \% = \frac{initial creep - creep after aging}{initial creep} \times 100$$

If the changing ratio is negative, the creep resistance is poor, which means that the aging resistance is poor.

The ingredients used in the reference example 0 and in the examples 1 to 5 are detailed as follows.

Component A: styrenic block copolymer (a):

A1: Vector 4411 (produced by ExxonMobil Chemical Company), which is star SIS (styrene-isoprene-styrene block co-polymer) with a styrene content of 40%

A2: YH1301, (produced by Yueyang Petrochemical Company), which is a linear SBS (styrene-butadiene-styrene block co-polymer) with a styrene content of 30%.

Component B: modified thermoplastic elastomer (b):

B1: TUFPRENE 912 (produced by ASAHI KASEI), which is a maleic anhydride modified SBS with a styrene content of 40%.

B2: ELVAX 40W (produced by DUPONT), which is a copolymer of ethylene-vinyl acetate with vinyl acetate content of 40 weight %.

Component C: wax (c)

C1: A-C 8 (produced by Honeywell), which is polyethylene wax with a drop melting point of 113° C.

C2: PMW (produced by ALPHAMIN), which is a microcrystalline wax with a drop melting point of 85° C.

C3: A-C680 (produced by Honeywell), which is a oxidized polyethylene wax with a drop melting point of 108° C.

Component D: Tackifier resin (d)

D1: Escorez 5600 (produced by EXXON CHEMICAL), which is a partly hydrogenated C9 modified dicyclic pentadiene (DCPD) petroleum resin with a ring and ball softening point of about 100° C.

D2: FP-125 (produced by Formosan Union Chemical Corp.), which are hydrogenated C9 petroleum resin with a ring and ball softening point of about 125° C.

Component E: plasticizer (e)

E1: KN4010 (produced by Karamay refinery), which is naphthenic oil with a viscosity of 140 mm²/s at 40° C.

E2: Primol N382 (produced by EXXONMOBIL CHEMICALS), which is a white mineral oil with a viscosity of 70 mm²/s at 40° C.

Component F: Antioxidant (f)

F1: Irganox 1010 (produced by CIBA), which is a hindered phenol antioxidant.

F2: Irganox 1076 (produced by CIBA), which is a hindered phenol antioxidant. Generally, the above two antioxidants are used in combination.

F3: Tinuvin P (produced by Sumitomo), which is a UV absorber.

The hot melt adhesive compositions of examples 1 to 5 of the present invention and the reference example 0 are prepared as follows. The weighted raw materials are sequentially added into an aluminum can whose temperature is controllable, the temperature is raised to 170° C., all the raw materials are melted and stirred till homogenous, the mixture is vacuum pumped and poured into a releasable silicon carton and then cooled. Example 0 uses a conventional elastic attachment hot melt adhesive as a reference sample.

The compositions of examples 0 to 3 are shown in table 1, the properties of the hot melt adhesive of examples 0 to 3 are shown in table 2. The hot melt adhesive compositions of table 1 and the properties of table 2 show that the addition of wax (c) and modified thermoplastic elastomer (b) can increase the elastic modulus of the hot melt adhesive significantly and thus improve the creep resistance thereof.

TABLE 1
the ratio of the raw materials in the
examples 0 to 3 (by weight percentage)

| Example | 0 | 1 | 2 | 3 |
|---|---|---|---|---|
| Component A/content | A1/23 | A1/6.5 | A1/10 | A1/16 |
| | | A2/10 | A2/6.5 | |
| Component B/content | / | B1/7.5 | B1/7.5 | B2/5.0 |
| Component C/content | / | C1/5.0 | C2/5.0 | C3/5.0 |
| Component D/content | D1/57 | D1/52 | D2/52 | D1/54 |
| Component E/content | E2/20 | E1/19 | E2/19 | E1/20 |
| Component F/content | / | F1/0.3 | F1/0.3 | F1/0.3 |
| | | F2/0.2 | F2/0.2 | F2/0.2 |
| | | F3/0.2 | F3/0.2 | F3/0.2 |

TABLE 2
the properties of examples 0 to 3

| Example | 0 | 1 | 2 | 3 |
|---|---|---|---|---|
| Viscosity at | 7800 | 8100 | 5990 | 6335 |
| 160° C., cps | | | | |
| G′ at 40° C., | 27890 | 460300 | 155200 | 279000 |
| Pa | | | | |
| Tg of the | 25.3 | 32.2 | 24.3 | 21.3 |
| adhesive | | | | |
| samples, ° C. | | | | |
| Strain at 5000 | 0.1256 | 0.0246 | 0.1115 | 0.0894 |
| Pa | | | | |
| Creep | 17.4 | 8.5 | 11.3 | 9.4 |
| resistance % | | | | |

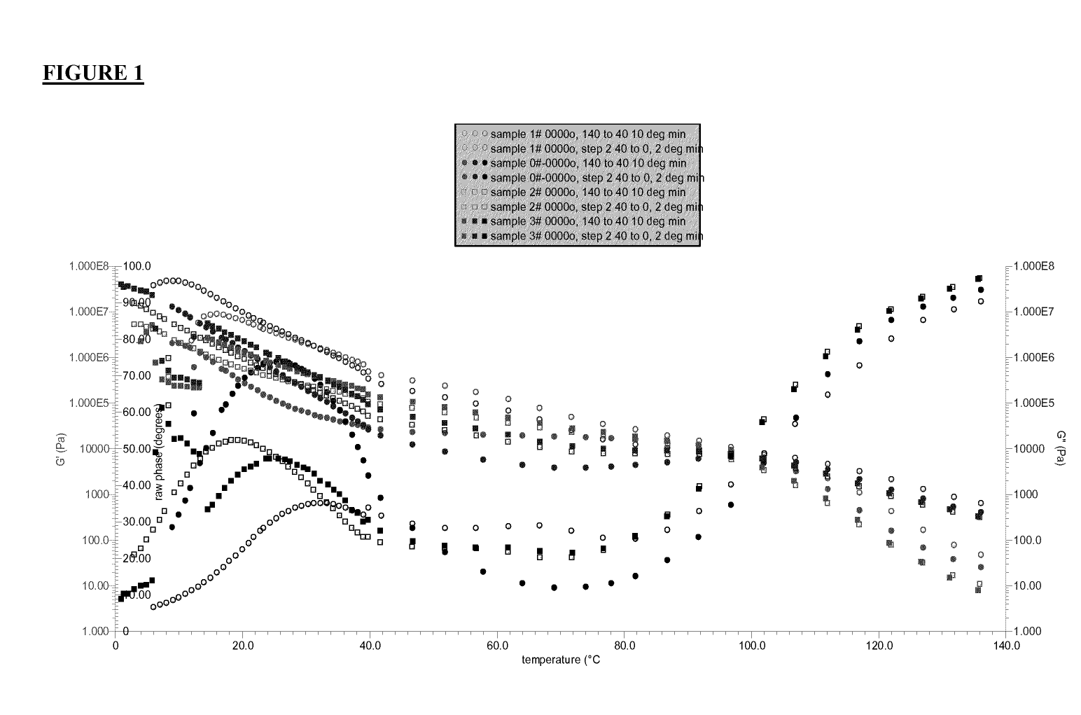

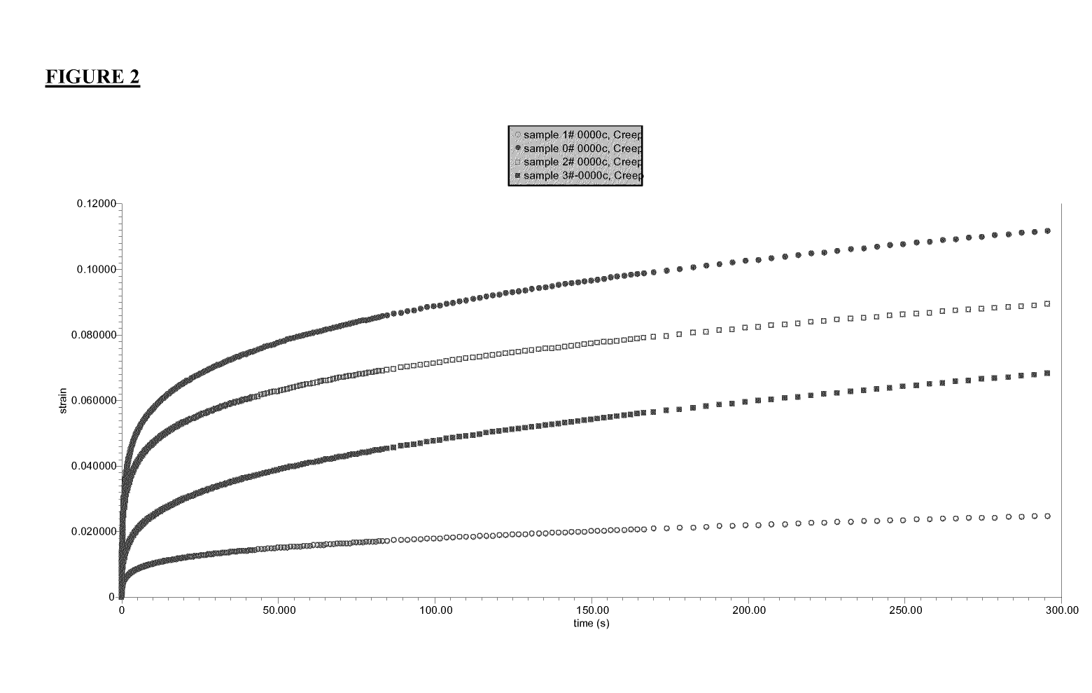

As can be seen from the combination of table 1, table 2, FIG. 1 and FIG. 2, compared with the conventional formulation of the reference example 0, the addition of wax (c) and modified thermoplastic elastomer (b) leads to a significant increase of the elastic modulus of the hot melt adhesive composition, the strain resistance under a certain stress is significantly improved and the creep resistance is excellent. Moreover, through the matching of various modified elastomers, various base polymers and various waxes, excellent thermal stability and homogeneous compatibility are obtained, such that the storage stability, i.e. the aging resistance of the disposable articles is ensured, which is verified by the following example 4 and example 5.

The formulations of examples 4 and 5 are shown in table 3; the properties of the hot melt adhesive of examples 0, 4 and 5 are shown in table 4. The hot melt adhesive compositions of table 3 and the properties results of table 4 show that: the addition of wax (c) and modified thermoplastic elastomer (b) can increase the elastic modulus of the hot melt adhesive significantly and thus improve the creep resistance thereof.

TABLE 3
the ratio of the raw materials in the examples
0, 4 and 5 (by weight percentage)

| Example | 0 | 4 | 5 |
|---|---|---|---|
| Component A/content | A1/23 | A1/7 | A1/7 |
| | | A2/11 | A2/10 |
| Component B/content | / | B1/5.0 | B1/5.0 |
| Component C/content | / | C1/5.0 | C2/10 |
| Component D/content | D1/57 | D2/54 | D2/51 |
| Component E/content | E2/20 | E1/18 | E2/17 |
| Component F/content | / | F1/0.3 | F1/0.3 |
| | | F2/0.2 | F2/0.2 |
| | | F3/0.2 | F3/0.2 |

TABLE 4
the properties of examples 0, 4 and 5

| Example | 0 | 4 | 5 |
|---|---|---|---|
| Viscosity at | 7800 | 5300 | 4458 |
| 160° C., cps | | | |
| G′ at 40° C., | 27890 | 302100 | 929500 |
| Pa | | | |
| Tg of the | 25.3 | 25.7 | 32.3 |
| adhesive | | | |
| samples, ° C. | | | |
| Strain at 5000 | 0.1256 | 0.075805 | 0.0081723 |
| Pa | | | |
| Initial creep | 17.4 | 9.0 | 6.5 |
| resistance % | | | |
| Creep resistance after | 23.8 | 6.8 | 5.7 |
| ageing for 4 weeks at | | | |
| 55° C. % | | | |
| Changing ratio of the | −37 | 24 | 12 |
| creep resistance % | | | |

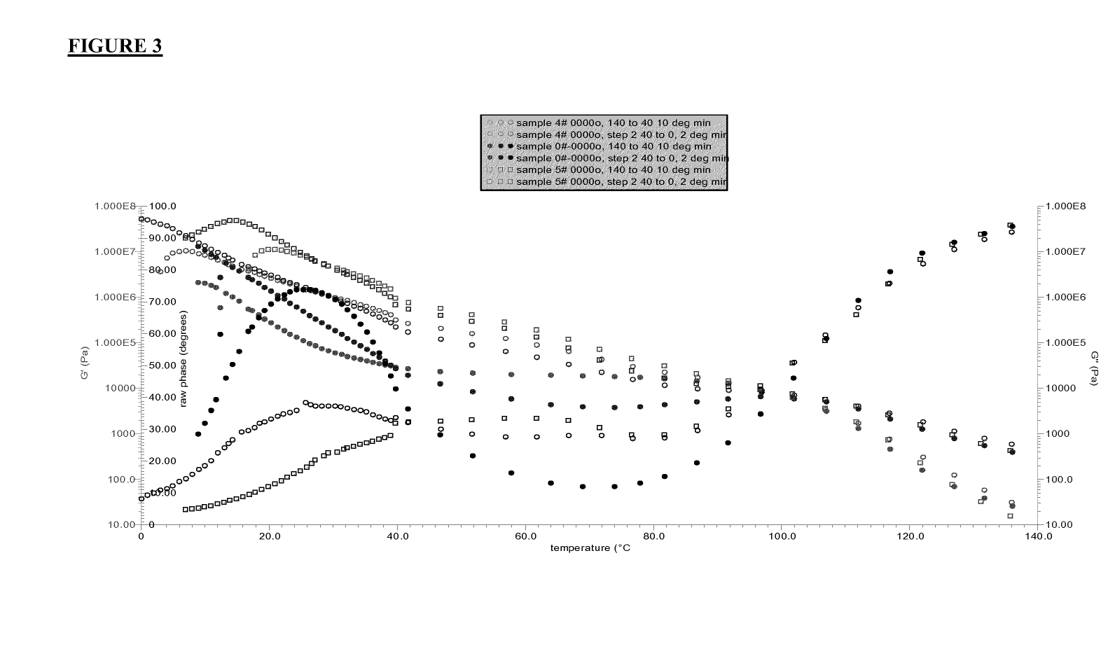

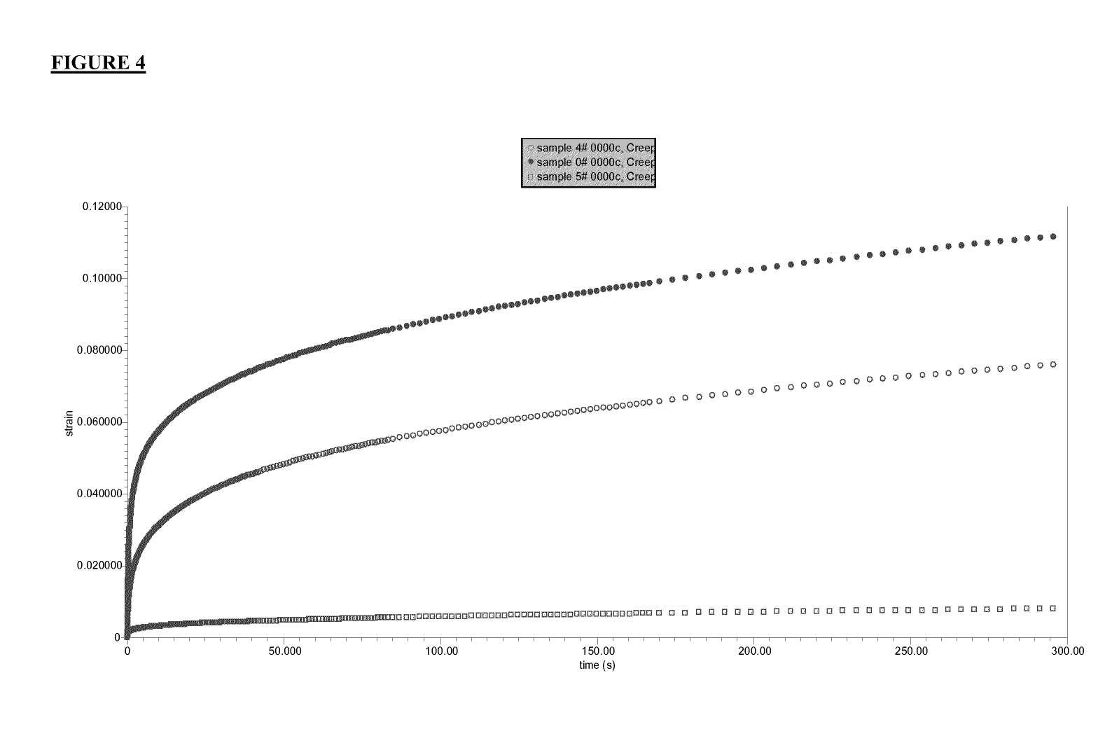

As can be seen from the combination of table 3, table 4, FIG. 3 and FIG. 4, compared with the conventional formulation of the reference sample, the addition of wax and modified elastomer polymer leads to the significant increase of the elastic modulus of the hot melt adhesive composition, the strain resistance under a certain stress is significantly improved and the creep resistance is excellent. Moreover, through the matching of various modified elastomers, various base polymers and various waxes, excellent thermal stability and homogeneous compatibility are obtained, the tendency of the migration of the small molecule plasticizer is changed by the wax, such that the storage stability is improved, i.e. the aging resistance of the disposable articles is excellent. Example 4 and example 5 also verifies that the creep resistance increases with the increasing of the wax content.

1. A composition comprising, based on the total weight of said composition, the content (expressed as weight percent) of the following components:
1 to 40% of one or more styrenic block copolymer (a) with a linear or star type structure and with the general formula: A-B-A
wherein: A is a homopolymer of styrene or vinyl toluene or other styrene derivatives
B is:
butadiene and/or isoprene;
partly hydrogenated isoprene, butadiene or isobutene;
substantially hydrogenated isoprene, butadiene or isobutene;
or mixtures thereof;
3 to 15% of a modified thermoplastic elastomer (b) comprising —COO— groups, including:
an acid or anhydride modified, linear or star type, styrenic block copolymer; and
an ethylene-vinyl acetate copolymer; and
5 to 25% of a wax (c), wherein said composition is a hot melt adhesive.

2. The composition of claim 1, wherein said copolymer (a) is styrene-isoprene-styrene (SIS), styrene-butadiene-styrene (SBS), styrene-isoprene/butadiene-styrene (SIBS), styrene-ethylene/butylene-styrene (SEBS) or styrene-ethylene/propylene-styrene (SEPS) or star co-polymer (SI)n or (SB)n, wherein n is equal to or greater than 3.

3. The composition of claim 1, comprising several copolymers (a) in combination, and a styrene content based on the total weight of styrene in the combined copolymers (a) of not less than 25%.

4. The composition of claim 1, wherein the modified thermoplastic elastomer (b) is an organic acid or anhydride modified.

5. The composition of claim 1, wherein the modified thermoplastic elastomer (b) is an ethylene-vinyl acetate copolymer with a vinyl acetate content equal to or greater than 40 weight %.

6. The composition of claim 1, wherein the wax (c) is paraffin, micro crystalline wax, polyethylene wax, polypropylene wax, by-product polyethylene wax, Fischer-Tropsch synthetic wax, various modified wax, or functionalized wax.

7. The composition of claim 1, wherein said composition further comprises 20 to 80 weight % of a tackifier resin (d).

8. The composition of claim 7, wherein the tackifier resin (d) has a ring and ball softening point comprised between 80 and 125° C.

9. The composition of claim 1, wherein said composition further comprises 5 to 40 weight % of a liquid plasticizer (e), wherein said liquid plasticizer is paraffinic oil, naphthenic oil, aromatic oil, low molecular weight polyisobutylene, tributyl phosphate, dioctyl phthalate, or a liquid hydrocarbon resin.

10. The composition of claim 1, wherein said composition comprises:
1 to 40 weight % of styrenic block copolymer (a);
3 to 15 weight % of modified thermoplastic elastomer (b);
5 to 25 weight % of wax (c);
20 to 80 weight % of tackifier resin (d);
5 to 40 weight % of liquid plasticizer (e); and
0.1 to 2 weight % of antioxidant (f).

11. A disposable absorbent article comprising a liquid permeable surface layer, a core layer for absorbing liquid and a liquid impermeable base layer, wherein said article comprises edge sealing elastic material bonded with the composition of claim 1.

12. The disposable absorbent article of claim 11, wherein the elastic material is natural rubber, synthetic rubber, polyurethane, polyester-polyurethane copolymer, polyacrylate, polyether ester, styrene block copolymers, or polyisoprene.

13. A process for manufacturing the disposable absorbent article of claim 1, comprising coating the hot melt adhesive composition:
on a substrate through spiral spraying, fiber coating, Omega spray, Surewrap™ spray, combed slot coating, slot coating, roll coating or painting, and
at a temperature selected in the range of from 140 to 180° C.

14. The process of claim 13, comprising coating through spiral spraying the hot melt adhesive composition on the elastic material as a strand.

15. The composition of claim 4, wherein the modified thermoplastic elastomer (b) is grafted, SBS, SIS, or SEBS.
